(19) United States
(12) Patent Application Publication
(10) Pub. No.: US 2010/0240134 A1
(43) Pub. Date: Sep. 23, 2010

(54) Beverage testing device and method

(76) Inventor: Christina Liner, Virginia Beach, VA (US)

Correspondence Address: Bradley D. Goldizen, HC 80 BOX 261, Maysville, WV, 26833, US

(21) Appl. No.: 12/383,061

(22) Filed: Mar. 18, 2009

(51) Int. Cl. G01N 33/02; G01N 31/22

(52) U.S. Cl. 436/20; 422/56

(57) ABSTRACT

The present invention is a testing device that comprises a plurality of indicated measurements printed onto a base or support material with inert ink. A reactive reagent is arranged above the indicated measurements and dissolves when introduced into a beverage according to the percentage of sugar present in the beverage. The visual indicator may be compared to a color chart to determine the amount of sugar present in the beverage. Otherwise, the testing device may indicate a numerical value of sugar content present in the tested beverage.

There are no related patent applications.

The subject matter of this application was not subject to federal research and development funding.

### TECHNICAL FIELD

Generally, the present invention relates to a beverage testing device that provides an indication of the presence of sugar and an amount of sugar present in a tested beverage. More specifically, a test strip indicator device comprises a rigid strip of inert material that is inserted into a drink to determine the presence of sugar and provide a visual indication of the amount of sugar in the beverage. The method comprises providing the device and dipping it into a beverage to determine whether the beverage contains sugar. A reference color chart may be provided for visually comparing an activated test strip to determine the amount of sugar present in the beverage. Otherwise, testing areas of the test strip may undergo a colorimetric change to reveal a number printed on a carrier material underlying a reagent deposited atop the number which corresponds to an amount of sugar present in a tested drink.

### BACKGROUND OF THE INVENTION

Diabetics and others who experience hypoglycemia must monitor the level of sugar and starches that they intake or consume. Such individual's bodies have trouble processing sugar. Consuming too much sugar may cause serious adverse reactions such as a coma or even death. Still other individuals who are on particular diets do not want to consume sugary products.

When purchasing food from restaurants, the purchaser may order a non-sugar containing drink or beverage. It is sometimes difficult to determine by taste whether the beverage contains sugar. Moreover, there is a real possibility that the incorrect soda tank has been installed in place of a non-sugar soda tank. Thus, there is need for a real time testing device that provides an indication of whether a beverage contains sugar at the served beverage temperature as well as a percentage of an amount of sugar present in the tested beverage.

U.S. Pat. No. 4,956,301 to Ismail et al., incorporated by reference thereto, discloses a test device and method of assaying blood or other liquid test samples for fructosamines. Fructosamines are found in the plasma of individuals. “Fructosamine” is the term used to describe proteins that have been glycated (ie, are derivatives of the non-enzymatic reaction product of glucose and albumin). Thus, the Ismail invention teaches detecting fructosamines in blood, plasma or other bodily food and fails to disclose a test that will indicate whether a beverage or liquid, prior to consumption, includes sugar.

U.S. Pat. No. 4,952,515 to Gleisner discloses a method of detection using a test strip having a non particulate dialyzed polymer layer. U.S. Pat. No. 6,557,484 to Engelman discloses a device that indicates the presence of sugar and/or caffeine in a beverage, incorporated by reference thereto. In Engelman, a drinking straw includes a sugar indicator section that changes color when inserted into a beverage that includes sugar. The Engelman device is an indicator only and does not include any assaying method or tool that indicates an amount of sugar present in a beverage. If a beverage includes only a small amount of sugar, the Engelmen indicator shows the same result as if the beverage contains a higher amount of sugar. Therefore, the Engelman is not useful in determining the content of sugar in a beverage.

### SUMMARY OF THE INVENTION

The invention is a calorimetric indicator strip that may be inserted into an unknown liquid to determine a presence of sugar and an amount of sugar present in the liquid. The indicator strip includes a support material having a reagent that reacts to sugar in a beverage to display an amount of sugar present in the beverage. The indicator strip is exposed to a beverage to be tested for a suitable period of time and a color change occurs if the sugar is present. Typically, the intensity of this change is proportional to the concentration of the sugar in the tested beverage. The color of the test strip is then compared to a known standard provided on a storage device that stores uncontaminated test indicator strips to enable a user to determine the amount of sugar present in the beverage. Otherwise, the indicator strip may include a plurality of percentage indicators that correspond to an amount of sugar present in the beverage. An overlay of material is deposited atop the percentage indicators. When dipped into a beverage having an unknown amount of sugar, the overlay of reactive material is dissolved or reacts to the sugar to indicate an amount of sugar present in the beverage.

The invention includes a carrier material having a plurality of test areas each comprising a percentage indicator or calorimetric region printed on the carrier material. A reagent material, and a possibly catalyst, are deposited atop each of the test areas and the percentage indicators and may be provided in different thicknesses such that a drink having a higher sugar level will cause a percentage indicator that displays a larger numeric value to be revealed after being dipped into a liquid having an unknown amount of sugar.

Otherwise, each test area of the test strips may include a calorimetric dye that indicates an amount of sugar depending on a color change. The reaction time between the indicators may be sped up by providing a different chemical formula or through varying the porosity of an overlay deposited atop the sugar indicating materials. The percentage indicators are overlaid by a reagent that reacts to sugar to provide a visual indicator that may be calorimetric or a numerical indicator.

For example, a reagent comprising enzymes glucose oxidase, peroxidase and a color indicator may be combined to indicate the presence of sugar in a drink. The reagent may be provided in test regions of a carrier strip to be reacted with sugar in a tested beverage to create a calorimetric device. A test strip is exposed to a beverage for testing. The visual results indicates the amount of sugar present in the tested beverage and may be compared to a color chart provided on a storage container to determine an amount of sugar present in the beverage. Otherwise, a contrasting color may be achieved when the reagent is exposed to sugar to cause a color change in the reagent deposited atop a printed numeric value which indicates an amount of sugar present in a tested drink.

In one instance, numeric values are printed in a test region on a carrier strip and include ink that is substantially the same color or hue as an initial reagent that overlays a test region. Sugar from a beverage is introduced to the reagent, preferably by dipping the carrier strip into the beverage, to cause the reagent to change color to a contrasting color to the color of the ink or a different hue to create a visual indicator. In this manner, the color of the underlying ink is revealed. That is, the exposed reagent contrasts with the underlying ink printed on the carrier material to indicate an amount of sugar present in a beverage.

The reaction undertaken begins when sugar is introduced to the reagent, by dipping the carrier into a drink to be tested and waiting an appropriate amount of time, typically a minute. Sugar reacts to the reagent causing a chemical reaction which provides a visual indication of an amount of sugar present in the drink. In one instance, the color change is a conversion of a color of an overlay that comprises a reagent and underlying numerical indicator printed on the carrier strip from a first color that is complementary to the numerical ink underlay. Before the indicator strip is exposed to sugar in the beverage, the underlying numerical value is hidden from view of the user. When exposed to sugar, one or more of the indicator regions of the indicator strip changes from the first color to a second color or different hue that contrasts with underlying ink to reveal one or more of the numerical values. The greatest numerical value revealed on the used device indicates the amount of sugar present in the drink.

It is an object of the invention to provide a test strip that includes a plurality of test regions that undertake a colorimetric change when exposed to sugar in a beverage to be tested. The test strip includes a plurality of reagent deposits arranged in the test regions to indicate the presence and amount of sugar in the tested beverage.

It is an object of the invention to provide a test strip that includes a plurality of test regions having reagent deposits arranged thereon such that a colorimetric change undertaken in the reagent deposits indicates whether sugar is present in a beverage to be tested and an percentage of sugar in the beverage. The test strip is exposed to the beverage and compared to a color chart provided on a container in which a plurality of test strips are provided.

It is an object of the invention to provide a test strip that includes a carrier material having a plurality of numerical indicia that corresponds to an amount of sugar present in a tested beverage. The numerical indicia comprise an inert ink of a specific color and a reagent having a complementary color to the color of the ink such that prior to expose to a sugar laden beverage, none of the indicia are visible. After exposure to a sugar laden beverage, the color of the reagent changes to a different color or hue that contrasts with the underlying ink to indicate an amount of sugar present in the beverage.

The above and further objects, details and advantages of the invention will become apparent from the following detailed description, when read in conjunction with the accompanying drawings.

### DESCRIPTION OF THE DRAWINGS

FIG. 1A shows a perspective view of a first embodiment of the invention. FIG. 1B shows an exploded view of the first embodiment of the invention.

FIG. 2A is a perspective view of a second embodiment of the invention. FIG. 2B shows an exploded view of the second embodiment of the invention. FIG. 2C shows an exposed test strip indicating that the percentage of sugar by concentration in the beverage is substantially between 15 and 24% 24% by volume.

FIG. 3 shows a container with a calorimetric chart for reading the indicated concentration of sugar in a tested beverage.

FIG. 4A shows a test strip being dipped into a beverage for testing purposes. FIG. 4B depicts the used test strip being compared to color chart provided on the storage container.

### DETAILED DESCRIPTION OF THE INVENTION

The embodiments of the invention and the various features and advantageous details thereof are more fully explained with reference to the non-limiting embodiments and examples that are described and/or illustrated in the accompanying drawings and set forth in the following description. It should be noted that the features illustrated in the drawings are not necessarily drawn to scale, and the features of one embodiment may be employed with the other embodiments as the skilled artisan recognizes, even if not explicitly stated herein. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims. Moreover, it is noted that like reference numerals represent similar parts throughout the several views of the drawings.

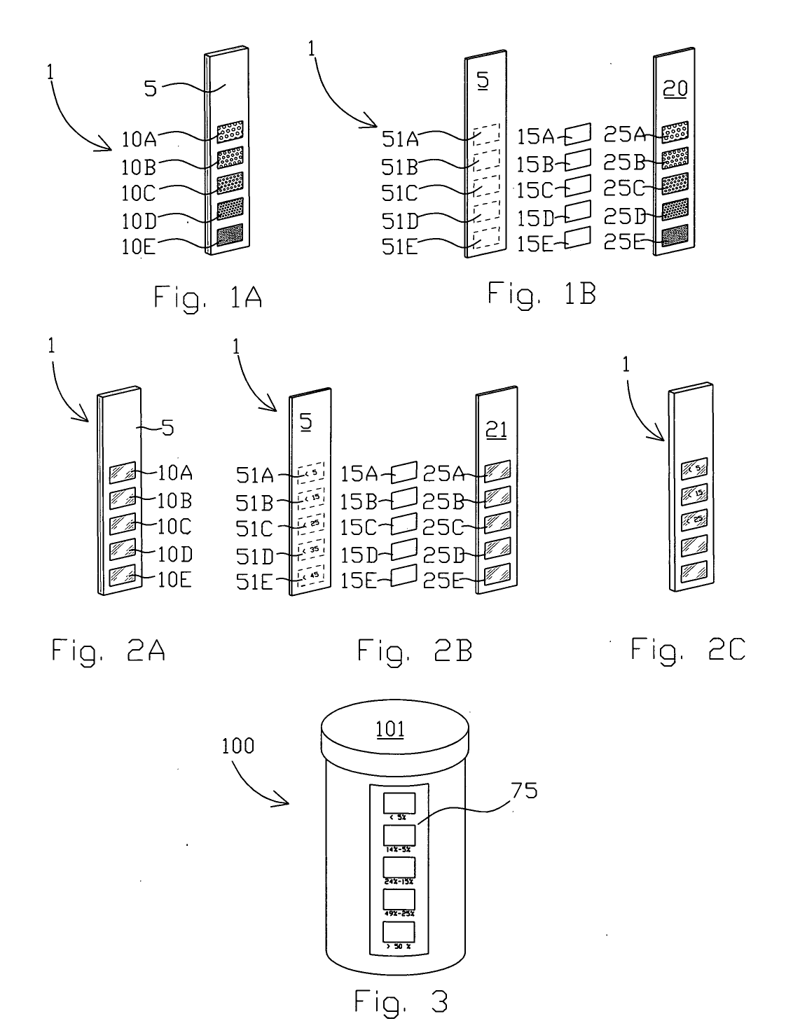

FIGS. 1A-1B shows a first embodiment of the invention wherein test strip 1 comprises a plurality of test regions 10A-10E. Each test region, proceeding from top to bottom, indicates a successively higher range for a sugar concentration in a tested beverage. The highest indicated level is the amount of sugar present in the tested beverage. As can be understood, a beverage having a larger amount of sugar will react to multiple test regions. For example, if a percentage of sugar in a particular beverage exceeds twenty-five percent, then multiple test regions may be activated. In a preferred embodiment, four of the five test regions indicate the presence of sugar. The fourth test region being the largest magnitude, the user recognizes that the amount of sugar present is between twenty-five and thirty-five percent. If no sugar is present in the tested beverage, then none of the test regions 10A-10E performs a calorimetric change and each of the test regions maintains its original color. If sugar is present in any concentration, there will be a colorimetric change in at least one of the test regions 10A-10E. As can be understood from these figures, the pore size of the overlay may vary from an absolute largest, as represented by window 25A to a smallest of 25E. The skilled artisan can recognize that chemical formulae may be changed such that uniform sized pores are provided or an inverse of the pore sizes above may be implemented. For example, the largest sized pores may be found in window 25E while the smallest pore size is provided in window 25A.

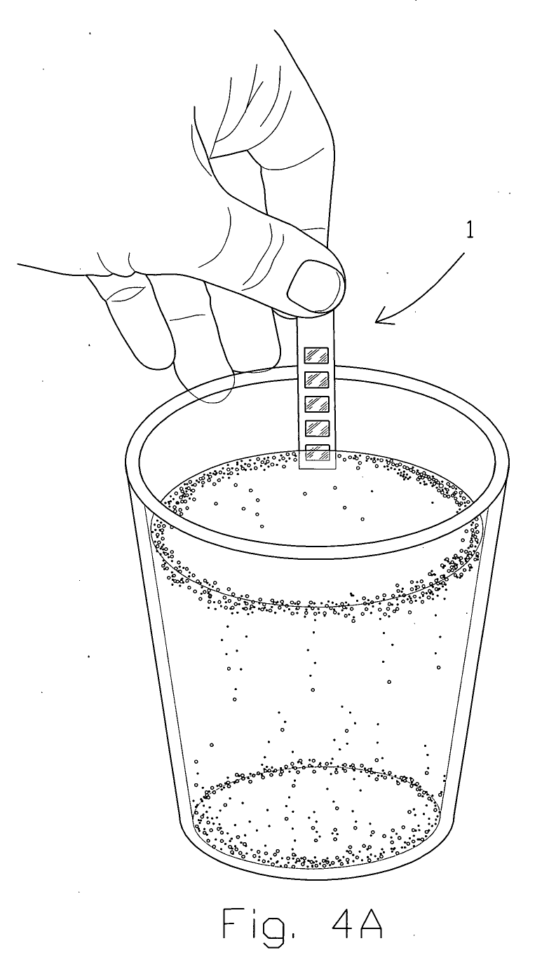

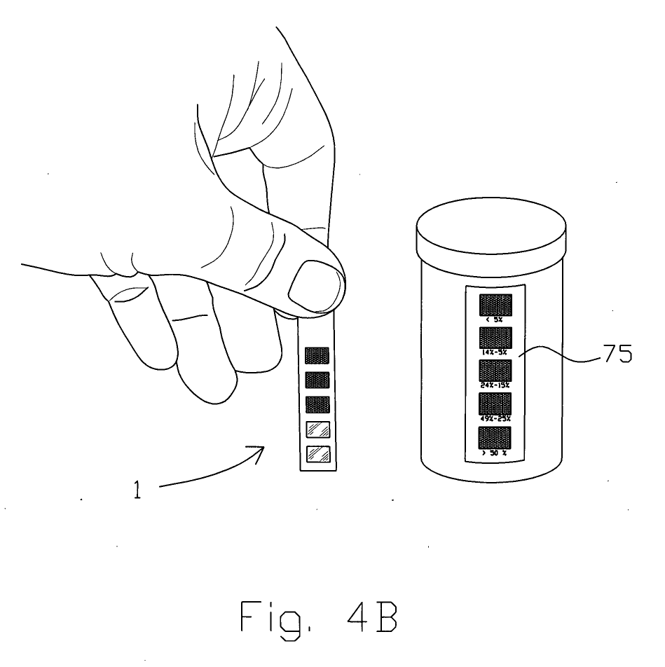

As can be recognized from FIG. 4B, a test strip 1 that has undertaken a calorimetric change is compared to a provided color chart on an adhesive strip 75. The color chart may be provided in a convenient container 100 that includes a cap 101. In this manner,

In this first embodiment, the test strip 1 includes a carrier material 5 that typically comprises an inert plastic. Carrier material 5 may be formed from one or more selected from a group consisting of polyethylene, polypropylene, polyvinyl chloride, and polyurethane and formed in a rectangular shape with an end that is easily grasped between a thumb and forefinger or two adjacent fingers such that it may be dipped into a beverage to be tested. The carrier material 5 comprises a plurality of test region areas 51A-51E onto which reagent deposits 15A-15E are arranged. The test regions are of uniform size and shape and are arranged equidistant from one another and centered between the longer sides of the carrier material 5. In this manner, a left margin and a right margin provided along the respective sides of each test area are equal. An overlay material 20 includes a plurality of windows 25A-25E that each comprises perforations. The overlay material 20 is preferably micro-porous such that liquid may enter into each window to react with the underlying reagent material without fear of any the reagent deposits 15A-15E being stripped from the carrier material. In one instance, the size of perforations in each window 25A-25E is unique to that particular window. Otherwise, the size of the perforations may be uniform with the number in each window varying. In either case, the perforations are large enough to allow a tested beverage to flow easily through the windows and onto the reagent deposits 15A-15E but small enough to retain the reagent deposits onto the carrier material 5. In this manner, the reagent deposits and any associated catalyst material is retained on the test strip when dipped into the beverage. Thus, the beverage is not contaminated by any reagent or catalyst during the testing procedure.

FIGS. 2A-2B depict a second embodiment of the invention. In this embodiment, a unique numeric value is printed inside each area defined by test region areas 51A-51E. The reagent deposits 15A-15E include individualized formulas such that a particular deposit undertakes a colorimetric change with a particular amount of sugar is present in tested beverage. Initially, the color of the reagent deposits 15A-15E is complementary in nature to the color of the ink used in printing the numeric values, such that when the deposits are arranged above the numeric values, the numeric values are hidden from view. When reacted with an appropriate amount of sugar, a particular reagent will undertake a calorimetric change to a contrasting color to the ink to reveal an amount of sugar present in a tested beverage. In this manner, the underlying ink becomes displayed when an appropriate amount of sugar is reacted with the reagent deposit of a particular test region 10A-10E.

In either embodiment, the size and number of perforations of the windows may be varied to manipulate the reaction time. Otherwise, chemical formulae may be individualized for each particular reagent deposit to provide the user with an upper range of the concentration of sugar present in a tested beverage. For instance, test region 10A may indicate a concentration of sugar that is less than 5%. That is, test region 10A will indicate the presence of any sugar in the beverage. Test region 10B indicates a concentration of sugar that is between 5% and 14% or less than 15%. Test region 10C indicates a concentration of sugar is between 15% and 24% or less than 25%. Test region 10D indicates a concentration that is between 25% and less than 35%. Test region 10E indicates a concentration that is greater than 45%. The overlay material 20, 21 prevents the reagent from being stripped off the carrier material when dipped into a beverage to be tested. In this manner, the user simply exposes the beverage to be tested onto the test strip and waits an appropriate amount of time. Thereafter, the amount of sugar present in the beverage is ascertained by determining the largest value displayed on the test strip. As can be recognized by the skilled artisan, lower values may also be displayed in this instance. However, the most accurate reading is the one displaying the largest amount of sugar.

### The Method

The method for displaying an amount of sugar present in a drink in the present invention comprises assembling a device as set forth above including providing a carrier material that includes a plurality of indicator regions. Each indicator region corresponds to an amount of sugar present in a tested drink. A reagent deposit is provided atop each indicator region. Each of the reagent deposits reacts to a specific amount of sugar present in the tested drink such that one of the reagent deposits tests for a lower amount of sugar than a successive reagent deposit. A porous overlay comprises a plurality of perforations that allow the drink to be exposed to each reagent deposit such that an amount of sugar present in the drink is displayed. The assembled test strip is dipped into the drink to cause a colorimetric change to be undertaken in one or more of the reagent deposits that indicates a concentration of sugar present in the drink.

The method may further comprise printing a unique numeric value on each indicator region wherein each unique numeric value corresponds to a particular amount of sugar present in the drink. The method may further also comprise providing a reagent that has a first and second color. The first color is complementary to a color of an inert ink used to print each unique numeric value onto each indicator region of the carrier material such that the unique numeric values are hidden from view until an appropriate amount of sugar is reacted with the reagent causing it to change to the second color which contrasts with the color of the inert ink to reveal a unique numeric value that corresponds to the amount of sugar in the tested drink.

As can be realized by viewing FIGS. 4A-4B, a test strip 1 is dipped into a beverage to be tested such that each of the test regions are exposed to the liquid. Thereafter, an appropriate amount of time is waited and the test strip 1 is viewed by the user. In FIG. 4B, the test strip 1 is then compared to the color chart 75 provided on the container 100. In this specific example, three of the test regions are colorimetricly reacted to indicate that an amount of sugar present in the tested beverage is substantially between 15% and 24%.

While the invention has been described with respect to preferred embodiments, it is intended that all matter contained in the above description or shown in the accompanying drawings shall be interpreted as illustrative and not in limiting sense. From the above disclosure of the general principles of the present invention and the preceding detailed description, those skilled in the art will readily comprehend the various modifications to which the present invention is susceptible. Therefore, the scope of the invention should be limited only by the following claims and equivalents thereof.

I claim:

1. A method for displaying an amount of sugar present in a drink, said method comprising:
providing a carrier material that includes a plurality of indicator regions, each indicator region corresponding to an amount of sugar present in a tested drink wherein a value of each indicator region is unique to that indicator region and different than all other indicator regions provided on the carrier material;
providing a reagent deposit atop each indicator region, each of said reagent deposits reacting to a specific amount of sugar present in the tested drink such that one of the reagent deposits tests for a lower amount of sugar than a successive reagent deposit;
providing a porous overlay comprising a plurality of perforations that allow the drink to be exposed to each reagent deposit such that an amount of sugar present in the drink is ascertained by viewing the reagent deposits, said porous overlay preventing each reagent deposit from being stripped from the carrier material when exposed to the drink;
dipping the carrier material into the drink to cause a colorimetric change to be undertaken in one or more of the reagent deposits that indicates a concentration of sugar present in the drink.

2. The method of claim 1 further comprising:
printing a unique numeric value on each indicator region wherein each unique numeric value corresponds to a particular amount of sugar present in the drink.

3. The method of claim 2 further comprising:
providing a reagent that has a first and second color, said first color is complementary to a color of an inert ink used to print each unique numeric value onto each indicator region of the carrier material such that the unique numeric values are hidden from view until an appropriate amount of sugar is reacted with the reagent causing it to change to the second color which contrasts with the color of the inert ink to reveal a unique numeric value that corresponds to the amount of sugar in the tested drink.

4. A test strip device for, prior to consumption, determining an amount of sugar present in a beverage or drink, said device comprising:
an inert base material that maintains structural integrity when dipped into a beverage;
a plurality of indicators printed onto the inert base material, said plurality of indicators comprising an inert ink having a first color and displaying a numeric value corresponding to a concentration of sugar present in a beverage;
a reagent arranged atop each indicator of said plurality of indicators and having a complementary color to said first color such that each numeric value is hidden from view until the reagent is reacted with sugar to display one or more of the numeric values such that an amount of concentration of sugar present in a tested beverage is displayed.

5. The test strip device of claim 4 further comprising
a catalyst arranged in said reagent such that said reagent dissolves when dipped into the beverage or drink containing sugar.

6. The test strip device of claim 4 wherein said inert base is formed in the shape of a rectangle.

7. The test strip device of claim 4 wherein said inert base is formed from one or more selected from a group consisting of polyethylene, polypropylene, polyvinyl chloride, and polyurethane.

8. A test strip that measures an amount of sugar present in a liquid, said test strip comprising:
a carrier material having a first face and formed in the shape of a test strip;
a plurality of test regions arranged along the first face, each test region proceeding from top to bottom, indicating a successively higher range for a sugar concentration in the liquid such that a highest indicated level is the amount of sugar present in the liquid;
an reagent indicator arranged atop each test region of the plurality of test regions, each reagent indicator having a unique reaction time to sugar that is different that a reaction time for any other reagent indicator such that different amounts of sugar will react to one or more of the reagent indicators to provide an accurate quantity of sugar present in the liquid; and,
an overlay having a plurality of windows, one window arranged atop of a respective reagent indicator and having pores through which the liquid may flow but that prevent the respective reagent indicator from separating from the carrier material.

9. The test strip of claim 8 further comprising:
a catalyst arranged in each of said reagent indicators dissolve when dipped into the beverage or drink containing sugar.

10. The test strip of claim 8 wherein said carrier material is formed in the shape of a rectangle.

11. The test strip of claim 8 wherein said carrier material is formed from one or more selected from a group consisting of polyethylene, polypropylene, polyvinyl chloride, and polyurethane.
